(19) United States
(12) Patent Application Publication
(10) Pub. No.: US 2024/0293488 A1
(43) Pub. Date: Sep. 5, 2024

(54) PHYSIOLOGICALLY ACCEPTABLE YEAST COMPOSITIONS AND USES THEREOF

(71) Applicant: AB Mauri (UK) Ltd, London (GB)

(72) Inventors: Carlos DE LECEA, Barcelona (ES); Jordi CUÑÉ CASTELLANA, Barcelona (ES); Maria TINTORÉ GAZULLA, Barcelona (ES)

(21) Appl. No.: 18/575,656

(22) PCT Filed: Jun. 30, 2022
(86) PCT No.: PCT/EP2022/068151

§ 371 Date: Dec. 29, 2023

(30) Foreign Application Priority Data
Jun. 30, 2021 (EP) 21382576.3
Feb. 11, 2022 (EP) 22382114.1

(51) Int. Cl. A61K 36/064; A61P 1/14; C12N 1/20; C12R 1/85

(52) U.S. Cl. CPC A61K 36/064; A61P 1/14; C12N 1/205; C12R 2001/85

(57) ABSTRACT

A physiologically acceptable composition comprising (i) at least one component selected from the group consisting of S. boulardii yeasts, S. boulardii lysates, S. boulardii cell wall components, and S. boulardii extracts, further comprising (ii) at least one component selected from the group of S. cerevisiae yeasts, S. cerevisiae lysates, S. cerevisiae cell wall components and S. cerevisiae extracts and further comprising (iii) at least one component selected from the group consisting of K. marxianus yeasts, K. marxianus lysates, K. marxianus cell wall components and K. marxianus extracts. The invention further relates to the composition for use as a medicament, as a food additive or functional ingredient for nutraceuticals, food for special medical purposes, cosmeceuticals and functional foods, and as a feed additive of functional ingredient in animal nutrition, as ingredient for topical application.

The invention relates to a physiologically acceptable composition, comprising yeast. The invention further relates to a method for preparing the composition. The invention further relates to the composition for use as a medicament, as a food additive or functional ingredient for nutraceuticals, food for special medical purposes, cosmeceuticals and functional foods, and as a feed additive of functional ingredient in animal nutrition, as ingredient for topical application. In particular, a composition according to the invention is suitable to maintain and improve gut health, for use in the treatment of a medical disorder, such as a disorder defined by pro-inflammatory markers or a gastro-intestinal disorder. A treatment of a medical disorder in accordance with the invention can be a preventive treatment or the treatment of an individual having said disorder.

Probiotics are live microorganisms, which confer a health benefit to the host, when administered in adequate amounts. Already for centuries, long before becoming aware of their potential health benefits, humans have benefitted from microorganisms in food, for example in fermented milk and yoghurt. Modem probiotics-containing nutrients and pharmaceuticals are direct derivatives of the early fermented food. To date, the most common probiotics are from the bacterial genera Lactobacillus and Bifidobacterium, however also yeasts are increasingly being considered as effective probiotic organisms.

Yeast-based probiotics are being recommended by several international guidelines to treat acute gastrointestinal disorders such as diarrhea or chronical conditions such as inflammatory bowel disease (IBD) and irritable bowel syndrome (IBS). The probiotic activities of these yeasts are considered multifactorial and includes improvement of gut barrier function, pathogen competitive exclusion, production of antimicrobial peptides, immune modulation, modulation of the microbiota and trophic effects. Yeast-based probiotics have many advantages over bacterial probiotics, such as a better withstanding of the extreme environments of the stomach to reach the intestines and an insensitivity to antibiotics providing the possibility to have probiotic effects during antibiotic treatment. Several yeast species have been shown to have a probiotic effect. Several yeasts have been suggested to act as probiotics such as some strains belonging to the genera Chrysonilia, Debaronmyces, Hanseniaspora, Kluyveromyces, Lachanencea, Metschnikowia, Pichia, Saccharonmyces, Torulaspora and Yarrowia (Ogunremi et al., 2015. J appl microbiol 117: 797-808; Sugiharto et al., 2018. J adv vet 5(3):332-342; Agarbati et al., 2020. Foods 9(3):287; Dufosse et aL., 2021. J Fungi 7(3): 177).

In order to address a need for novel strains of microorganisms which may exert a beneficial effect on health preventively and/or curatively on either specific pathologies or dysfunctions or on both physical and psychic general health conditions, US2010/303778 provides a specific Saccharonmyces cerevisiae strain (deposited at the Collection Nationale de Cultures de Microorganismes under No. CNCM I-3856) and a specific a Saccharomyces var. boulardii yeast strain (deposited at the Collection Nationale de Cultures de Microorganismes under No. CNCM I-3799). Said yeasts may e.g. be used as in compositions for treating an intestinal disorder.

There is a continuing need to provide alternative treatment possibilities of medical disorders, in particular gastro-intestinal disorders, such as diarrhea, IBD or IBS, or disorders wherein inflammatory makers play a role, such as rheumatoid arthritis, osteoarthritis, topical dermatitis, psoriasis, allergy or obesity. There is further a continuing need to provide alternative products suitable for improving gut health or improving gastro-intestinal functioning. It is in particular an object to provide a composition, which is suitable to provide an improved protection of the gut barrier function or anti-inflammatory effect compared to a known probiotic composition, comprising a yeast such as described in the above discussed prior art. One or more further objects that may be addressed will follow from the description herein below.

We have now found that one or more of said objects are realized by providing a specific composition comprising at least three different yeasts.

Accordingly, the invention relates to a physiologically acceptable composition comprising (i) at least one component selected from the group consisting of S. boulardii yeasts, S. boulardii lysates, S. boulardii cell wall components, and S. boulardii extracts, further comprising (ii) at least one component selected from the group of S. cerevisiae yeasts, S. cerevisiae lysates, S. cerevisiae cell wall components and S. cerevisiae extracts and further comprising (iii) at least one component selected from the group consisting of K. marxianus yeasts, K. marxianus lysates, K. marxianus cell wall components and K. marxianus extracts.

Further, the invention relates to said physiologically acceptable composition according to the invention for use as a medicament.

Furthermore, the invention relates to said physiologically acceptable composition for use as in the treatment of a human or animal by therapy.

Furthermore, the invention relates to the physiologically acceptable composition according to the invention for use in maintaining or improving of gut health or gastrointestinal functioning.

Furthermore, the invention relates to the physiologically acceptable composition according to the invention for use in the treatment of an individual with a gastrointestinal disorder. Preferred gastrointestinal disorders to be treated are disorders related to the impairment of the gut barrier function. In a particularly preferred embodiment, the composition according to the invention is for use in the treatment of diarrhea, IBD or IBS.

Furthermore, the invention relates to the physiologically acceptable composition according to the invention for use in the treatment of an individual with a disorder defined by one or more pro-inflammatory markers, in particular one or more markers selected from the group consisting of IL-8, IP-10, MCP-1, TNFα and TNFα/IL-10. Regarding, IL-10, it is observed that this is modulatory/anti-inflammatory compound, of which the concentration may be reduced and result in an increased TNFα/IL-10 ratio, also if the TNFα itself does not increase; this ratio is also a relevant maker for a disorder defined by one or more pro-inflammatory markers.

Preferably, a disorder defined by one or more pro-inflammatory markers to be treated in accordance with the invention is selected from the group consisting of rheumatoid arthritis, osteoarthritis, topical dermatitis, psoriasis, allergy and obesity.

Furthermore, the invention relates to a use of a physiologically acceptable composition as a food additive, a feed additive, a functional food in human nutrition, a functional food in animal nutrition, a food additive or functional ingredient for nutraceuticals.

Furthermore, the invention relates to a use of a physiologically acceptable composition as a probiotic, a postbiotic, a paraprobiotic, a prebiotic, a symbiotic or a probiotic-substitute.

Furthermore, the invention relates to a medical device comprising a physiologically acceptable composition.

A (medical) use according to the invention can be a preventive treatment (prophylactic) or a treatment of an individual, in particular a human, having a medical disorder to be treated. A treatment of an individual having a medical disorder can comprise curing the medical disorder, reducing suffering, alleviating or relieving one or more symptoms associated with said medical disorder.

The effectivity of a prophylactic treatment can routinely be determined, e.g. by comparing cohorts or test animals treated with a composition for use according to the invention and a reference product (placebo), with a reduced incidence. The effect of a treatment of an individual having the disorder can be a complete cure, an alleviation of a symptom, reduced suffering etc.

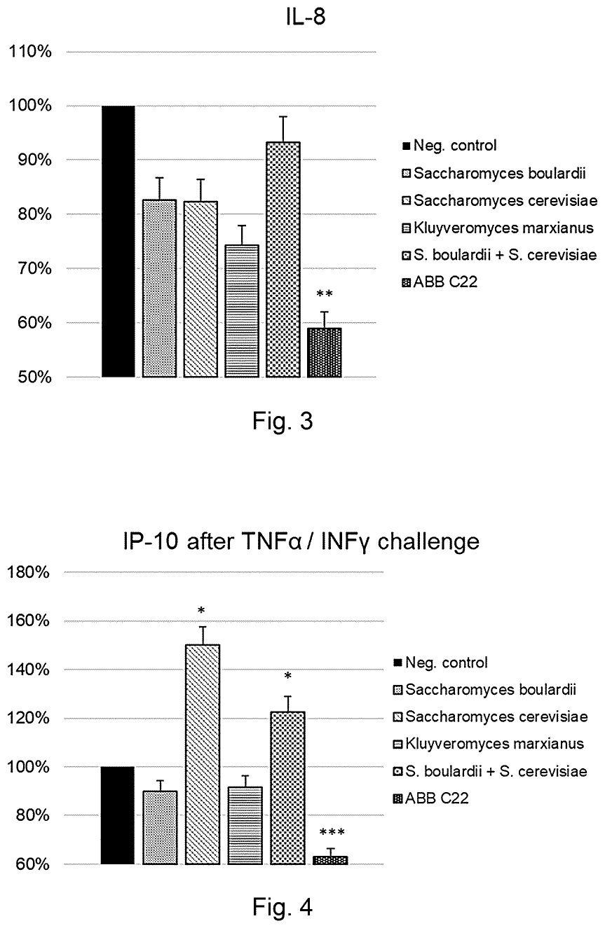

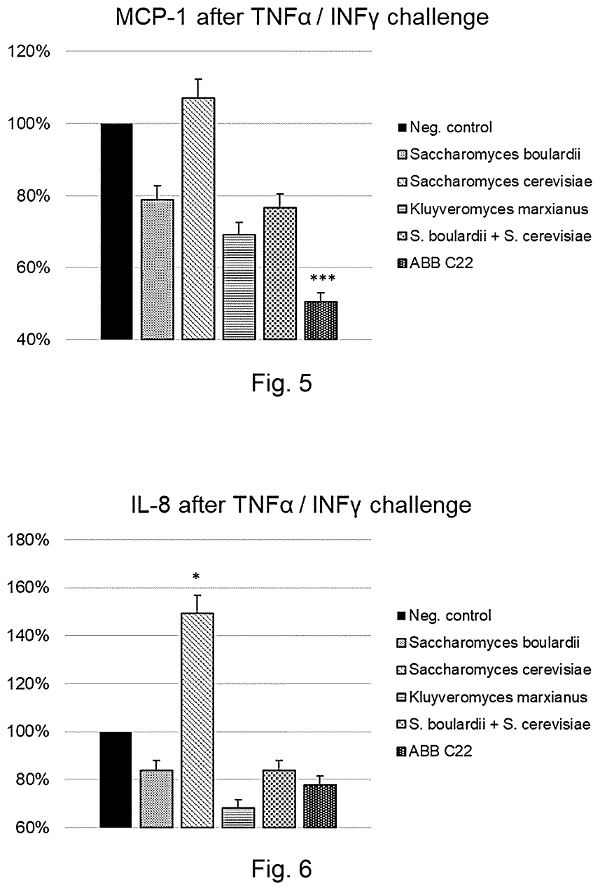

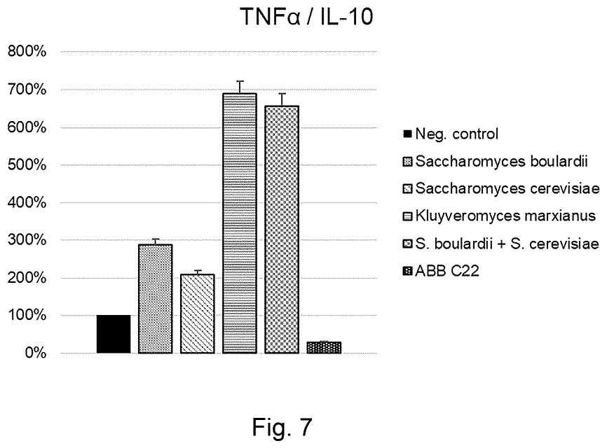

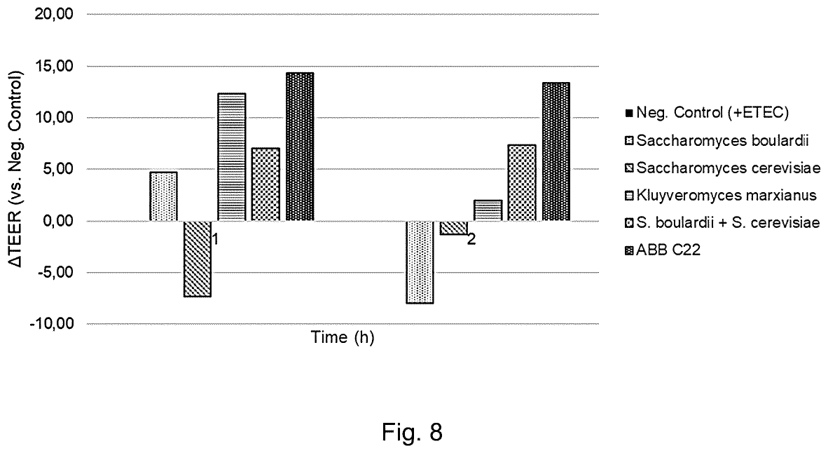

As illustrated in the Examples, below, a composition according to the invention has been found effective in improving one or more relevant markers for gut health, such as a reduction of pro-inflammatory cytokine production by gut epithelial cells and immune cells (i.e. indicating an anti-inflammatory effect) and an increase of trans epithelial electric resistance (i.e. indicating an improved protection of the gut barrier function). The effect on these markers are indicative of a positive effect for use in the treatment of gastro-intestinal disorders characterized by inflammation and/or a loss of epithelial integrity, like diarrhea, IBD or IBS. Further, the results are supporting that the composition is also effective for the treatment or prevention of a gastrointestinal disorder, such as diarrhea or a gastrointestinal infection, because simulating an acute gastrointestinal infection by using a pro-inflammatory stimulus or infecting epithelial cells with bacteria that can induce diarrhea, such as a typical diarrhea causing E. coli strain, several markers for gut health improve. In particular, a surprising effect is obtained by combining three yeasts: S. boulardii, S. cerevisiae and K. marxianus, In the examples, it is illustrated how several markers for gut health are improved by such combination whilst individual yeasts are ineffective, less effective, or even have an adverse effect. Synergy is, amongst others, illustrated for MCP-1 (FIG. 2), IL-8 (FIG. 3), for IP-10 and MCP-1 after a TNF-alpha/INF-gamma challenge (FIGS. 4 and 5) and for the ratio TNF-alpha/IL-10 (FIG. 7). Furthermore, it is illustrated how said yeast combination improves gut epithelium integrity, measured by the increase in trans epithelial electric resistance (TEER) over a gut cell monolayer (FIGS. 8, 9 and 10).

Said yeasts or part thereof can each independently be selected from viable yeast cells and non-viable yeast cells. As illustrated by the Examples these yeasts do not need to be viable. Accordingly, the inventors further conclude that one, two or each of said S. boulardii, S. cerevisiae and K. marxianus in the composition can be replaced fully or in part by a yeast lysate, cell wall material or a yeast extract of respectively S. boulardii, S. cerevisiae and K. marxianus

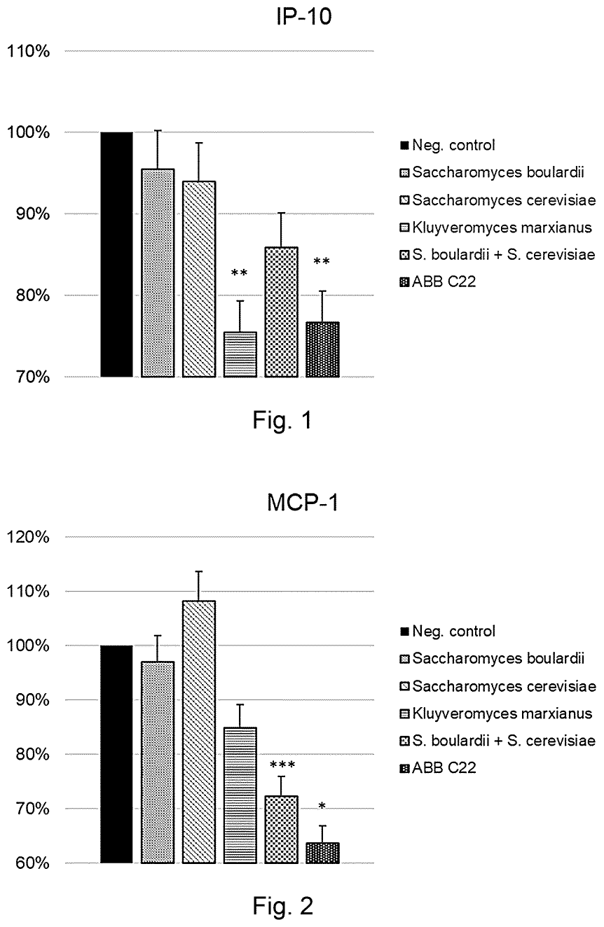

FIG. 1 depicts the in vitro IP-10 chemokine production by Caco-2 cells after incubation with 3 yeasts and a combination thereof, in the absence of a pro-inflammatory stimulus.

FIG. 2 describes the in vitro production by Caco-2 cells of MCP-1 chemokine after incubation with 3 yeasts and a combination thereof, in the absence of a pro-inflammatory stimulus.

FIG. 3 describes the in vitro production by Caco-2 cells of IL-8 chemokine after incubation with 3 yeasts and a combination thereof, in the absence of a pro-inflammatory stimulus.

FIG. 4 depicts the in vitro IP-10 chemokine production by Caco-2 cells after incubation with 3 yeasts and a combination thereof, in the presence of a pro-inflammatory stimulus (TNF-α/INF-γ) simulating an inflamed gut epithelium.

FIG. 5 describes the in vitro production by Caco-2 cells of MCP-1 chemokine after incubation with 3 yeasts and a combination thereof, in the presence of a pro-inflammatory stimulus (TNF-α/INF-γ) simulating an inflamed gut epithelium

FIG. 6 describes the in vitro production by Caco-2 cells of IL-8 chemokine after incubation with 3 yeasts and a combination thereof, in the presence of a pro-inflammatory stimulus (TNF-α/INF-γ) simulating an inflamed gut epithelium.

FIG. 7 shows the in vitro reduction in TNFα/IL-10 ratio observed in human THP-1 cells (macrophages).

FIG. 8. depicts the protective effect on the gut epithelium by the different yeasts and combinations thereof. A comparison of the yeasts versus the negative control is shown at 1 hour (bars at the left) or 2 hours (bars at the right) of incubation with an infective agent known to disrupt the epithelium monolayer (E. coli ETEC H10407). Gut epithelium integrity is measured by the increase in trans epithelial electric resistance (TEER) of the monolayer.

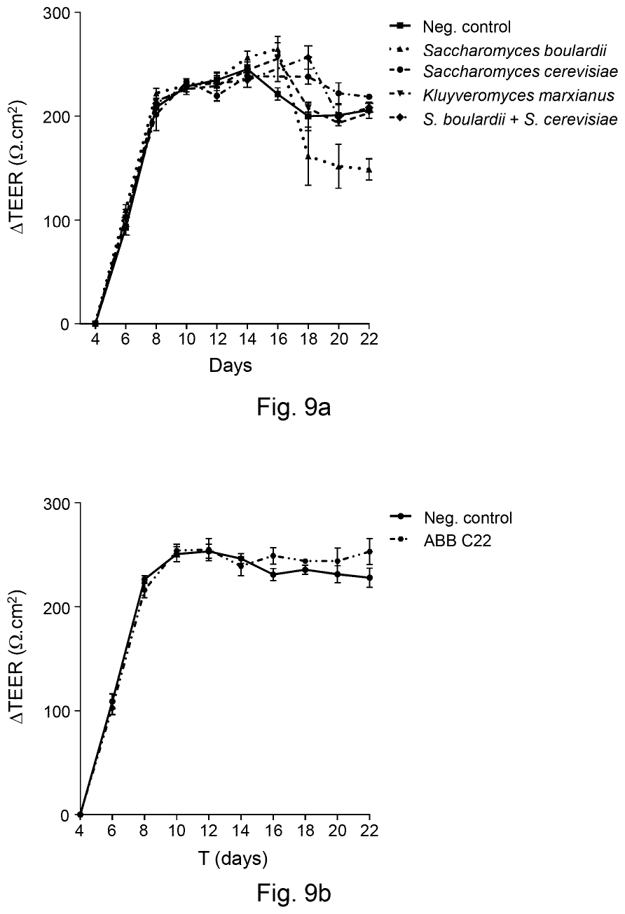

FIG. 9 shows the differentiation of the gut epithelium measured by an increase in TEER when incubating the Caco-2 cells monolayer in the presence of the yeasts (top) and their combination (bottom). Note that some error bars are too little to be visible.

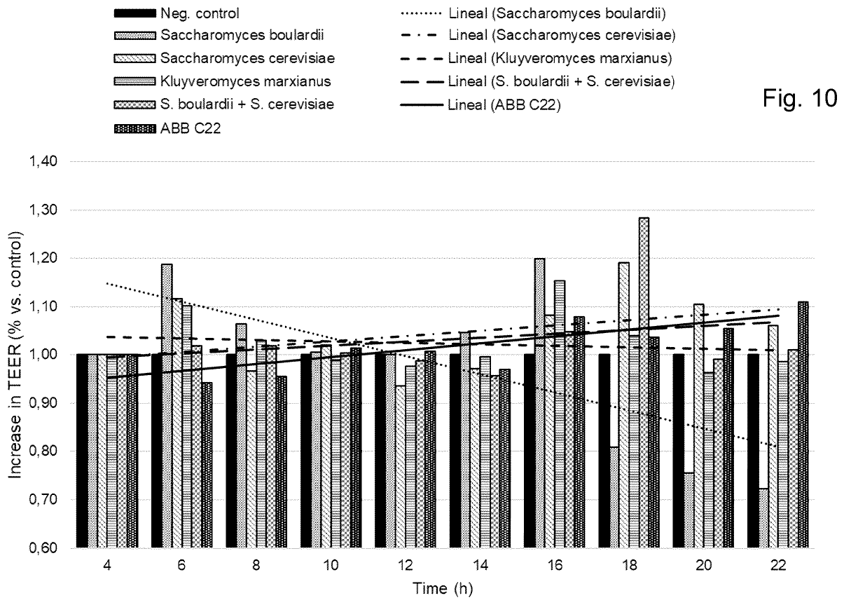

FIG. 10 compares the effect observed in FIG. 9 (differentiation of the gut epithelium measured by an increase in TEER when incubating the Caco-2 cells monolayer in the presence of the yeasts and their combination) by means of % of increase vs. the control. Regression formulae for the trend lines are as follows, S. boulardii: y=−0.0374x+1.1848; S. cerevisiae: y=0.011x+0.9844; K. marxianus:y=−0.0031x+1.0408: S. boulardii+S. cerevisiae: y=0.0081x+0.9871; and ABB C22: y=0.0142x+0.9384.

For the purpose of clarity and a concise description, features are described herein as part of the same or separate embodiments, however, it will be appreciated that the scope of the invention may include embodiments having combinations of all or some of the features described.

As used herein, the singular forms “a”, “an” and “the” are intended to include the plural forms as well. The term “or” includes any and all combinations of one or more of the associated listed items, unless the context clearly indicates otherwise (e.g. if an “either . . . or” construction is used). It will be understood that the terms “comprises” and “comprising” specify the presence of stated features but do not preclude the presence or addition of one or more other features. It will be further understood that when a particular step of a method is referred to as subsequent to another step, it can directly follow said other step or one or more intermediate steps may be carried out before carrying out the particular step, unless specified otherwise.

The term “(at least) substantial(ly)” is generally used herein to indicate that it has the general character or function of that which is specified. When referring to a quantifiable feature, this term is generally used to indicated that it is more than 50%, in particular at least 75%, more in particular at least 90%, even more in particular at least 95% of the maximum of that feature.

The term ‘essentially free’ is generally used herein to indicate that a feature is not present or present in such a low amount that it does not significantly affect the property of the product.

In the context of this application, the term “about” means generally a deviation of 15% or less from the given value, in particular a deviation of 10% or less, more in particular a deviation of 5% or less.

As is used herein, the term “physiologically acceptable composition”, refers to a composition that is suitable for administration to an individual such as an animal or a human.

As is used herein, the term “probiotics”, refers to live microorganisms, which when administered in adequate amounts, confer a health benefit to an individual. Probiotics include all sorts of microorganisms including bacteria and yeasts.

As is used herein, the term “inactivated”, or “dead”, or “non-viable”, refers to an organism, such as a yeast, not being capable of reproduction or colonization. An inactivated organism can have intact or broken cell membranes. The skilled person will be able to obtain an inactivated organism yeast based on common general knowledge and the information disclosed herein. Possible means include irradiation, heat inactivation, sonication, lyophilization and chemical inactivation.

As used herein, the term “heat-killed”, refers to an organism inactivated by heat-treatment and such not capable of metabolic activity nor colonization. Means of heat-treatment to inactivate an organism are known to a person skilled in the art and include tyndallization, pasteurization, ultra-high temperature (UHT) heating, Ohmic heating (or Joule heating), blanching, drying, boiling and sterilization.

As is used herein, the term “tyndallization”, refers to a sterilization process often used to heat-kill probiotic microorganisms. Tyndallization involves repeating a heat-killing step for a certain consecutive days, for example as described on page 11 in the Handbook of Microbiological Media (third edition, 2004, CRC Press) by Ronald M. Atlas. Therefore, the term “tyndallized”, refers to an organism having been heat-killed by tyndallization and such not capable of metabolic activity or colonization.

As used herein, the term “cell lysis” refers to any type of cell disruption that results in the release of intercellular biological components naturally contained in the cells of an organism. Therefore, the term “lysate”, refers to a product obtained after cell lysis. A “lysate”, as used herein, means in particular essentially the entire lysate obtained by lysis of an organism and therefore comprises macromolecules such as DNA, RNA, proteins, peptides and lipids from the lysed cell; as well as cellular debris, including cell wall material, and cell membrane components from the lysed cells. Methods for obtaining a lysate are known to a person skilled in the art and include enzymatic, physical and chemical methods. Cell wall components can be separated from the fluid part of the lysate e.g. by centrifugation of filtering.

As used herein, the term “extract” of a yeast, refers to a fluid part or fraction of the yeast cell or lysate, in particular liquid contents of yeast cells, in particular obtainable by filtration or by centrifugation, or a fraction thereof, obtainable by extraction from the cells or the lysate, using an extracting phase,

As used herein, the term “metabolite”, refers to any substance derived from the growth or maintenance of the yeast, persisting in the culture medium and with no need to be preserved by special techniques. Examples of metabolites are organic and inorganic acids, proteins, (poly)peptides, amino acids, (co)enzymes, fatty acids, (esterified) lipids, carbohydrates (including monosaccharides, disaccharides and polysaccharides), lipoproteins, glycolipids, glycoproteins, sugar phosphates, vitamins, salts, metals, or nucleic acids.

As is used herein, the term “alive”, or “viable”, refers to an organism being capable of reproduction or colonization.

As is used herein, the term “individual”, refers to any living organism such as an animal or human that can benefit from the administration of a physiologically accepted composition of the invention. The term “animal” as used herein, refers in particular to vertebrate animals including fish, birds, mammals, reptiles and amphibians. The animal can be a farm animal, domestic animal or laboratory animal. An individual, which may be treated in accordance with the invention may in particular be selected from humans, nonhuman primates and monkey species, cows, sheep, pigs, goats, horses, dogs, cats, rodents such as mice, rats and guinea pigs, poultry, such as chickens, hens, turkeys, ducks and geese and aquatic animals such as fish and shrimp. The term individual does not denote a particular age or sex. (such as male/female). Thus, humans of any age group, including adults (18 years of age and older) and children (0-17 years of age), e.g. newborn individuals (0-12 months of age), toddlers (12-36 months of age), can be treated in accordance with the invention. In a preferred embodiment, the composition is for use of the treatment of a human, for which the examples in particular illustrate a beneficial, even synergistic, effect on intestinal cells. In another preferred embodiment, the composition is for use in the treatment of a non-human mammal.

As is used herein, the term “nutritional product”, refers to a composition intended for ingestion by an individual providing at least one nutrient to the individual. Nutritional products generally comprise one or more components selected from the group consisting of protein, fat, carbohydrate and micro-nutrients.

As is used herein, the term “nutraceutical”, refers to any nutritional product providing extra health benefits in addition to the basic nutritional value found in foods.

As is used herein, the term “cosmeceutical”, refers to any cosmetic product with bioactive ingredients purported to have health benefits.

As is used herein, the term “oral rehydration salt (ORS)”, refers to a saccharide-based salt solution suitable for use in oral rehydration therapy. ORS are recommended in the prevention of dehydration from diarrhea from any cause and in individuals of any age. ORS's are further recommended to treat a dehydrated individual of any age.

As used herein, the term “prebiotic” refers to any substance that is selectively utilized by microorganisms conferring a health benefit to an individual. Prebiotics are in particular nondigestible food ingredients that stimulate the growth and/or activity of said microorganisms.

As used herein, the term “postbiotic” refers to any preparation of inactivated microorganisms and/or their components that confers a health benefit to an individual. The components that confer a health benefit may be a mixture of metabolic products secreted by probiotics in cell-free supernatants, such as enzymes, secreted proteins, short chain fatty acids, vitamins, secreted biosurfactants, amino acids, peptides, organic acids, etc.

As used herein, the term “paraprobiotic” refers to inactivated microorganisms and/or cell fractions that confers a health benefit to an individual.

As used herein, the term “synbiotic” or “symbiotic”, refers to any preparation comprising a mixture of probiotics and prebiotics.

Hereinbelow, the term “yeast material” or “yeast based fraction” is used as a genus for living yeast cells, inactivated yeast cells, yeast lysates, yeast cell wall components and yeast extracts. Likewise, the term “microbiological material” is used as a genus for living microorganisms, inactivated microorganisms, lysates of microorganisms, cell wall components of microorganisms and extracts of microorganisms.

### Components of a Physiologically Acceptable Composition

In this invention, the S. boulardii can be any strain classified or classifiable as S. cerevisiae var. boulardii, in particular any such strain that is probiotic in a living or inactivated form. In an embodiment, the composition comprises at least one S. boulardii selected from the group of a S. boulardii DSM 33954, a S. boulardii CNCM 1-745, S. boulardii Hansen CBS 5926, S. boulardii BLD-3, S. boulardii CCTCC M2012116, S. boulardii CNCM 1-1079, S. boulardii ATCC MYA-796, S. boulardii Unique28, S. boulardii Kirkman, S. boulardii Unisankyo and S. boulardii CNCM 1-3799. In particular good results have been achieved with a composition comprising S. boulardii DSM 33954.

In this invention, the S. cerevisiae can be any strain belonging to the species S. cerevisiae, in particular any strain that is probiotic in a living or inactivated form, with the exception of strains classified or classifiable as S. cerevisiae var. boulardii. The species S. cerevisiae is also referred to in the art as baker's yeast, brewer's yeast or Candida robusta. In an embodiment, the composition comprises at least one S. cerevisiae selected from the group of a S. cerevisiae Y1529 (as deposited at ATCC), a S. cerevisiae CNCM I-3856 S. cerevisiae S288C and S. cerevisiae UFMG 905. S. cerevisiae S288C and S. cerevisiae Y1529 are particularly preferred S. cerevisiae. Good results have been achieved in particular with a mineral-enriched S. cervisiae, such as zinc-enriched S. cerevisiae.

In this invention, the K. marxianus can be any strain belonging to the species K. marxianus, This species is also referred to in the art as Saccharomyces marxianus, Candida Kefyr, Candida pseudotropicalis, Kluyveromyces fragilis, Kluyveromyces cicerisporus. In an embodiment, the composition comprises one or more strains selected from the group consisting of K. marxianus AS41, K. mmarxianus B0399, K. marxianus CIDCA 8154, K. marxianus CBS1553, K. marxianus M3, K. marxianus V21/012435 and K. marxianus Z17. Good results have in particular be achieved with a composition according to the invention comprises K. marxianus V21/012435 cells. Accordingly, K. marxianus V21/012435 cells, an extract thereof, a lysate thereof or cell wall material thereof is present in a preferred composition of the invention

A person skilled in the art will be able to determine if a given yeast strain is classified or classifiable as S. cerevisiae var. boulardii, as species S. cerevisiae or as K. marxianus by using standard references such as e.g. “The yeasts, a taxonomic study” (CP Kurtzman, J W Fell and T Boekhout), 5^{th} edition, 2011, Elsevier. Furthermore, person skilled in the art will be able to differentiate S. cerevisiae var. boulardii from other yeast strains belonging to the species S. cerevisiae based on standard references such as e.g. Edwards-Ingram L, Gitsham P, Burton P, et al., “Genotypic and physiological characterization of Saccharomyces boulardii, the probiotic strain of Saccharomyces cerevisiae”, Appl Environ Microbiol 2007; 73: 2458-67.

The relative amounts of the (i) at least one component selected from the group consisting of S. boulardii yeasts, S. boulardii lysates, S. boulardii cell wall components and S. boulardii extracts (also referred to herein as S. boulardii-based fraction), the (ii) at least one component selected from the group of S. cerevisiae yeasts, S. cerevisiae lysates, S. cerevisiae cell wall components and S. cerevisiae extracts (also referred to herein as S. cerevisiae-based fraction) and the (iii) at least one component selected from the group consisting of K. marxianus yeasts, K. marxianus lysates, K. marxianus cell wall components and K. marxianus extracts (also referred to herein as K. marxianus-based fraction) can vary within wide limits.

Generally, the S. boulardii-based fraction of the composition according to the invention is: 0.05-99.95 wt. %, preferably 5-95 wt. %, more preferably 15-80 wt. %, in particular 20-60 wt. %, more in particular 25-50 wt. %, based on total yeast components.

Generally, the S. cerevisiae-based fraction of the composition according to the invention 0.05-90 wt. %, preferably 5-90 wt. %, more preferably 10-90 wt. %, more preferably 15-80 wt. %, in particular 20-80 wt. %, more in particular 20-60 wt. %, more in particular 25-50 wt. %, based on total yeast components.

Generally, the K. marxianus-based fraction of the composition according to the invention is: 0.05-99.95 wt. %, preferably 5-90 wt. %, more preferably 10-90 wt. %, more preferably 15-80 wt. %, in particular 20-80 wt. %, more in particular 20-60 wt. %, more in particular 25-50 wt. %, based on total yeast components.

Usually, the S. boulardii-based fraction, the S. cerevisiae-based fraction and the K. marxianus-based fraction form together at least 10 wt. % of the total microbiological material (such as bacterial, algae or fungal cells, lysates thereof, extracts thereof), preferably at least 25 wt. %, more preferably at least 50 wt. %, in particular at least 75 wt. %. If desired, other microbiological material may be present in the composition, in particular a probiotic micro-organism or cell material of a probiotic micro-organism. Thus, the total of the S. boulardii-based fraction, the S. cerevisiae-based fraction and the K. marxianus-based fraction is 100% of the total microbiological material or less, for instance 99 wt. % or less, based on total microbiological material.

In an advantageous embodiment, the physiological composition comprises 5-95 wt. % S. boulardii-based fraction, 10-80 wt. % S. cerevisiae-based fraction and 5-95 wt. % K. marxianus-based fraction (all based on based on total yeast components and preferably on total microbiological material). In particular, good results are achieved, e.g. with respect to several pro-inflammatory markers or TEER with a composition having a S. boulardii-based fraction content in the range of 15-50 wt. %, a S. cerivisieae-based fraction content in the range of 15-50 wt. % and a K. marxianus-based fraction content in the range of 15-50 wt. %, all based on of the total microbiological material of the composition, with the proviso that the total of said three fractions is 100 wt. % or less. As the skilled person will understand, based on the information disclosed herein and common general knowledge, different contents may be applied with satisfactory results.

Since safety issues with the use of live microorganisms have been arisen, for example in fragile or immunocompromised patient groups or neonates, interest in using non-viable inactivated probiotics has increased. Several inactivation methods are known to a person skilled in the art. and include irradiation, heat inactivation, sonication, lyophilization and chemical inactivation. Tyndallization is a sterilization process often used to heat-kill probiotic microorganisms. Interestingly, in accordance with the invention tyndallized yeasts have shown to exert relevant biological responses such as restoring the normal intestinal homeostasis.

Accordingly, advantageously, the physiologically acceptable composition of the invention comprises at least one inactivated yeast selected from S. boulardii, S. cerevisiae and K. marxianus. Preferably inactivated S. cerevisiae, inactivated S. boulardii and inactivated K. marxianus are present in the composition. If one or more of said yeasts is a mineral-enriched yeast, such as a zinc-enriched yeast, it is in particular preferred that the mineral-enriched yeast is inactivated. Preferably, the inactivated yeast is heat-killed. More preferably, the inactivated yeast is tyndallized. The tyndallization may be based on tyndallization processes generally known in the art. In particular, good results have been achieved with a composition comprising a tyndallized S. boulardii, further a tyndallized S. cerevisiae and further a tyndallized K. marxianus Alternatively, or in addition, the physiologically acceptable composition of the invention may comprise at least one live yeast selected from S. boulardii, S. cerevisiae and K. marxianus. If at least one of said yeast is present in an alive form, preferably at least K. marxianus is present in an alive form.

The S. boulardii as included in the physiologically acceptable composition of the invention for administration into the gastrointestinal tract is usually present in a concentration ranging from 10⁶ cells per gram (based on total weight of the yeast components) to 10¹¹ cells per gram (based on total weight of the yeast components). preferably from 10⁷ cells per gram (based on total weight of the yeast components) to 10¹¹ cells per gram (based on total weight of the yeast components), more preferably 10⁹ cells per gram (based on total weight of the yeast components) to 2×10¹⁰ cells per gram (based on total weight of the yeast components). The S. cerevisiae as included in the physiologically acceptable composition of the invention for administration into the gastrointestinal tract is usually present in a concentration ranging 10⁶ cells per gram (based on total weight of the yeast components) to 10¹¹ cells per gram (based on total weight of the yeast components). preferably 10⁸ cells per gram (based on total weight of the yeast components) to 5×10⁹ cells per gram(based on total weight of the yeast components). The K. marxianus as included in the physiologically acceptable composition of the invention for administration into the gastrointestinal tract is usually present in a concentration ranging from 10⁶ cells per gram (based on total weight of the yeast components) to 10¹⁰ cells per gram (based on total weight of the yeast components). preferably from 10⁷ cells per gram (based on total weight of the yeast components) to 5×10⁹ cells per gram (based on total weight of the yeast components).

S. boulardii as included in the physiologically acceptable composition of the invention for another mode of administration, such as topical administration, is usually present in a concentration ranging from 10⁴ cells per gram (based on total weight of the yeast components) to 10¹¹ cells per gram (based on total weight of the yeast components), preferably 10⁶ cells per gram (based on total weight of the yeast components) to 10¹⁰ cells per gram (based on total weight of the yeast components). S. cerevisiae as included in the physiologically acceptable composition of the invention for another mode of administration, such as topical administration, is usually present in a concentration ranging from 10⁴ cells per gram (based on total weight of the yeast components) to 10¹¹ cells per gram(based on total weight of the yeast components). preferably 10⁶ cells per gram(based on total weight of the yeast components) to 10¹⁰ cells per gram (based on total weight of the yeast components). K. marxianus as included in the physiologically acceptable composition of the invention for another mode of administration, such as topical administration, is usually present in a concentration ranging from 10⁴ cells per gram (based on total weight of the yeast components) to 10¹¹ cells per gram (based on total weight of the yeast components), preferably 10⁶ cells per gram (based on total weight of the yeast components) to 10¹⁰ cells per gram (based on total weight of the yeast components).

Concentrations of live and inactivated yeasts are measured and expressed in cells per gram total yeast components. For live yeasts the concentration in ‘cells per gram’ is equivalent as ‘colony forming units (CFU) per gram total yeast components. If a combination of live cells and inactivated cells (not colony forming) are present’, the total of cells is will usually be in the above mentioned usual range, preferably in an above mentioned preferred range or more preferred range. For a yeast lysate, for an extract or for cell wall components a suitable concentration usually corresponds to the amount of lysate, extract respectively cell wall components obtainable from 10⁴ cells per gram (based on total weight of the yeast components) to 10¹¹ cells per gram (based on total weight of the yeast components).

The physiologically acceptable composition of the invention may comprise a yeast, in particular a S. cerevisiae, that is mineral-enriched. Preferably, the physiologically acceptable composition of the invention comprises a zinc-enriched yeast, more preferably a zinc-enriched S. cerevisiae. The physiologically acceptable composition of the invention may comprise a mineral salt, preferably zinc salt, most preferably a zinc sulphate.

Zinc-enriched yeast provide a natural source of highly bioavailable zinc. Zinc is considered a key nutrient for immunity and diarrhea management. Interestingly, it has been shown that zinc supplementation as organically-bound or blended zinc via yeast organisms results in a better bioavailability compared to inorganic zinc. Not only zinc, but several other minerals such as selenium, chromium, iron, copper, magnesium, manganese, potassium, calcium and iodine have been shown to be beneficial in restoring an individuals' mineral balance and can be enriched to yeasts to become a better bioavailability. A mineral-enriched yeast can be obtained by culturing a yeast in a medium in which one or more minerals are added, such as zinc, selenium, chromium, iron, copper magnesium, manganese, potassium, calcium or iodine. The mineral typically binds organically to yeast protein or is otherwise absorbed, resulting in yeasts having the one or more minerals absorbed in their cells. The minerals can be added to the medium before, during or after culturing. As used herein, a mineral-enriched yeast strain, is in particular a yeast strain fermented in the presence of a mineral salt or to which a mineral salt has been added after fermentation, containing a final concentration of such mineral up to 12 wt. % of the dry wt. % of the whole product, in particular up to 5 wt. %, more in particular up to 2 wt. %, e.g. up to 1 wt. %. For zinc addition to a final concentration in the range of 1-12 wt. %, in particular of about 4 wt. % to about 10 wt. % is preferred.

### Administration Mode and Formulations of a Physiologically Acceptable Composition

The physiologically acceptable composition of the present invention is preferably administered into the gastrointestinal tract or topically. Administration into the gastrointestinal tract is preferably orally. In a specific embodiment, the composition is administered by tube feeding or as a suppository. Topical administration in particular includes administration to a mucus or dermal administration. Specific examples are administration the eye and vaginal administration. Formulations of a composition according to the invention suitable for oral intake include but are not limited to: capsules, coated capsules, tablets, sachets, pills, pearls, softgels, vials, powders, granules, solutions, suspensions, emulsions, elixirs, syrups, sprays, lozenges, troches, gums, hard candies and gels. Formulations of a composition according to the invention suitable for topical administration include creams (e.g. for vaginal application), capsules (e.g. for vaginal application), tablets (e.g. for vaginal application), ointments, pastes, foams, gels, lotions, shampoo, mousse, sprays (e.g. for application on skin, eye or mucosa), suppository, solutions (e.g. for vaginal application), plaster, bio adhesives, liquids (e.g. for application on skin, eye such as eye drops or mucosa) and suspensions.

Formulations of a composition according to the invention suitable for topical administration include compositions suitable for application to a part. of the gastro-intestinal tract at which the effect of the yeast components is needed, such as a suppository or a gastro-intestinal medical device.

Topical administration in the treatment of a gastrointestinal disorder generally comprises administration at mucosa or epithelium of the gastro-intestinal tract. A medical product comprising the composition (for use) according to the invention can be a product suitable to create a protective biofilm or the like at the surface of the gut epithelium. Such product can be based for instance on known products for treatment of IBS.

In a specific embodiment, the composition is to be administered as a sustained release product.

The physiologically acceptable composition of the present invention can be administered using a medical device. Such medical device for topical administration of the physiologically acceptable composition, may be a plaster, a (transdermal) patch.

The composition according to the invention may be a food product. The food product may be a fermented food product or a non-fermented food product. Examples of a particularly suitable food products include dairy products such as a yogurt, a yogurt drink, cheese, milk, milk powder, infant formula, cream, ice-cream, cream powder and butter: fruit-based products such as fruit juice, compote or fruit jelly; solid foodstuffs such as flours, cereals, a snack, a biscuit, and liquid formulations such as vegetable beverages, smoothies, isotonic drinks, salts solutions and enteral nutrition recipes. The physiologically acceptable composition according to the invention for intake by an animal may be any appropriate food product for animals and include, in addition to the food products listed above, tablets, coated tablets, granules, cereal grains, (dried) meat, (dried) fish, oil meals, cakes, cookies, sugarcane, and roughages such as grasses, hays, silage, root crops, straw and stover.

The nutritional product according to the invention may be in the form of a dietary supplement, a food additive, a feed additive, a functional food in human nutrition, a functional food in animal nutrition, a food additive or functional ingredient for nutraceuticals or food for special medical purposes.

The physiologically acceptable composition according to the invention can be a pharmaceutical product, a cosmeceutical product or a nutraceutical product. Said pharmaceutical product may further comprise a pharmaceutically acceptable adjuvant and/or excipient. Adjuvants and excipients are well known to a person skilled in the art.

An oral hydration salt. (ORS) is a preferred example of a product according to the invention; an ORS which may be classified as a pharmaceutical (e.g. WHO) or as a food supplement, dependent on national regulations. The ORS preferably contains S. boulardii yeast cells, S. cerevisiae yeast cells and K. marxianus yeast cells, of which advantageously at least a substantial part is inactivated. Particularly suitable yeast cells for an ORS have been found to be S. boulardii DSM 33954 (available as ABB1 from ABBiotek—Spain, h=tps>://www.abbot/k.com), S. cerevisiae Y1529 (as deposited at ATTC, available from ABBiotek as ABB6) and K. marxianus V21/012435 (as deposited at the National Measurement Institute, Port Melbourne Vic 3207, Australia, available from ABBiotek as ABB7). A mixture of these three yeast strains is available from ABBiotek as ABB C22

The physiologically accepted composition may further comprise a bulking agent, in particular a carbohydrate, for example maltodextrin.

An example of an ORS formulation in liquid format comprises S. boulardii, S. cerevisiae and K. marxianus (ABB C22); and further water, glucose, sodium citrate, sodium chloride, maltodextrin, zinc sulphate, silicon dioxide), flavor, sweetener (acesulfame K) and acidifier (citric acid).

An example of an ORS formulation in powder format comprises S. boulardii, 10 K. marxianus and S. cerevisiae (ABB C22); and further dextrose, lemon, flavour, citric acid, magnesium citrate, malic acid, sodium citrate, sodium chloride, potassium phosphate, calcium ascorbate, sucralose and riboflavin.

### The Production of a Physiologically Acceptable Composition

The physiologically acceptable composition can be made by combining the different components based on methodology known per se for making yeast preparation.

For instance, all yeasts may be produced from a non-GMO yeast strain. A fermentation process known per se for the yeast of interest can be used to produce a primary grown, yeast whose growth occurs under aseptic, aerobic conditions. The resulting product, or yeast cream may be held in refrigerated storage to maintain cell viability, if desired.

The yeast cream can be subjected to an inactivation treatment, such as a high temperature sterilization or tyndallization to obtain a heat-treated version of the yeast. If desired, the yeast can be dried, for instance by spray drying, which preferably is done after inactivation (if non-viable yeast is to be used for the composition).

Maltodextrin or other bulking agents can be used as support agent to have standardized concentrations. The three yeasts are mixed, which mixing may be followed by a homogenization step.

### Methods of Treating an Individual and Improving an Individual's Gut Health or Gastrointestinal Functioning

In accordance with the invention, the physiologically acceptable composition is advantageously used in the treatment of an individual with a gastrointestinal disorder, such as irritable bowel syndrome (IBS); an inflammatory bowel disorder (IBD), for instance Crohn's disease or ulcerative colitis; functional constipation; diarrhea, for instance antibiotic associated diarrhea, traveler's diarrhea, acute gastroenteritis, pediatric diarrhea, dysbiotic diarrhea or chronic diarrhea, in particular in immunodeficient patients; functional abdominal pain; functional bloating, Postprandial Distress Syndrome; gastrointestinal allergy or intolerance; necrotizing enterocolitis; gastrointestinal infections caused by bacteria, such as Escherichia, Salmonella, Shigella, Staphvlococcus, Vibrio, Campylobacter, Yersina, Clostridium or Helicobacter; gastrointestinal infections caused by a virus, such as norovirus, adenovirus, cytomegalovirus, enterovirus astrovirus, hepatitis virus or rotavirus; gastrointestinal infections caused by a parasite, such as Giardia, Entamoeba, Cryptosporidium, Cyclospora or Ascaris; and combinations thereof. Preferably, a gastrointestinal disorder selected from the group consisting of IBD, IBS and diarrhea is treated. In a specific embodiment, the diarrhea is a bacterially induced diarrhea, e.g induced by E. coli.

Further, the physiologically acceptable composition is advantageously used in the treatment of an individual with a disorder defined by one or more pro-inflammatory markers, said markers include IL-8, IP-10, MCP-1, TNFα/IL-10 and TNFα. Disorders defined by pro-inflammatory markers that are treated by the invention include rheumatoid arthritis, osteoarthritis, topical dermatitis, psoriasis, allergy and obesity. In particular, the physiologically acceptable composition of the invention is suitable for the treatment of inflammatory disorders defined by one or more of said pro-inflammatory markers, in the absence of infection.

Further, the physiologically acceptable composition according to the invention is particularly suitable to maintain or improve gut health or gastrointestinal functioning, especially those conditions involving impairment or weakness of the gut barrier function or alterations in the inflammatory cytokines release. The term ‘gut health’ as described herein means the health status of the gut. The gut health status of an individual might be affected by, for example, infections causes or non-infectious causes, such as a non-optimal diet. The term “gastrointestinal functioning” refers to the operation of all of the organs and structures associated with the gastrointestinal system. Markers to determine gut health or gastrointestinal functioning are known to a person skilled in the art and include for example trans epithelial electrical resistance as an indication of epithelial barrier integrity and markers for inflammation and injury. Examples of markers are described in Celi et al. (2019) “Biomarkers of gastrointestinal functionality in animal nutrition and health” Animal Feed Science and Technology 250: 9-31.

The administration dosage, duration and frequency can be chosen within wide limits, dependent on the intended purpose and the subject to whom the composition is to be administered. The duration of treatment can be a relatively short period, e.g. of a week or less, or a day or less, e.g. for an acute manifestation of a disorder or symptom of a disorder, e.g. diarrhea. The duration of a treatment can also be prolonged, e.g. for a week or more, a month or more or a year or more, e.g. in case of a chronic disorder such as IBD.

The physiologically acceptable composition for use according to the invention is may be administered as a single dosage for a complete treatment, dependent on the application. If multiple dosages are intended, the number of dosages is generally 10 times per day or less. E.g. in case of the use of an ORS more than 3 dosages per day may be administered, but typically it is administered about 3 time per day or less, preferably about 2 times per day or less, in particular about once per day or less. In an embodiment, the composition is administered (on average) at least about once a week. Preferably, it is administered (on average) at least once per period of three days, more preferably (on average) at least once per period of two days.

The physiologically acceptable composition may comprise or can be co-administered with a(nother) probiotic, a prebiot ic, a postbiotic, an antibiotic, an analgesic, an anti-inflammatory agent, an anti-diarrheal agent such as an inhibitor of motility or secretion, an(other) oral rehydration salt, a laxative or a mixture thereof.

A person skilled in the art will be able to determine an appropriate dose of the physiologically acceptable composition to administer to an individual based on common general knowledge and the information disclosed herein without undue experimentation. As the skilled person will understand, an actual preferred dosage depends on a variety of factors, including the activity of the specific yeasts employed, the metabolic stability and length of action of that yeast, the age, body weight, general health, sex and species of the individual diet, mode and time of administration, rate of excretion, drug combination, the severity of the particular disorder, and the individual. The dosages disclosed herein are indicative of an average case for a human individual. There can of course be individual instances where higher or lower dosage ranges are merited. The usual effective daily dose for oral administration or other administration into the gastrointestinal tract, in particular for humans, is from about 100 mg (of the yeast components) to about 1000 mg (of the yeast components), preferably, from about 200 mg (of yeast components) to about 900 mg (of the yeast components), more preferably, from about 200 mg (of the yeast components) to about 650 mg (of yeast components). The usual effective daily dose for topical administration, in particular for humans, is from about 1 mg (of yeast components) to about 1000 mg (of the yeast components), preferably from about 5 mg (of the yeast components) to about 500 mg (of the yeast components).

The invention provides a physiologically acceptable composition for use as a medicament.

The invention provides a method of treating an individual in need of an improvement of gut health or in need of an improvement of gastrointestinal functioning, comprising the administration of an effective amount of the physiologically acceptable composition according to the invention, hereby improving gut health or gastro-intestinal functioning. The invention has in particular found to be effective in improving the gut barrier function and in driving an anti-inflammatory status, as well as in the protection from viral, bacterial and yeast infection, while modulating the microbiota towards an eubiotic state.

The invention provides a method of treating an individual with a gastrointestinal disorder, such as diarrhea, IBD, IBS or (other) gastrointestinal disorders related to the impairment of the gut barrier function, comprising the administration of an effective amount of the physiologically acceptable composition according to the invention to said individual.

The invention provides a method of treating an individual with a disorder defined by pro-inflammatory markers such as rheumatoid arthritis, osteoarthritis, topical dermatitis, psoriasis, allergy or obesity, comprising the administration of an effective amount of the physiologically acceptable composition according to the invention to said individual.

The invention provides the use of the physiologically acceptable composition according the invention for use in the preparation of a product, which may be a medicament or a food product (such as a medical or clinical food), for use in the treatment of a gastrointestinal disorder, preferably a gastrointestinal disorder selected from the group consisting of diarrhea, IBD and IBS or a(nother) gastrointestinal disorder related to the impairment of the gut barrier function.

The invention provides the use of the physiologically acceptable composition according the invention for use in the preparation of a product, which may be a medicament or a food product (such as a medical or clinical food), for use in the treatment of a disorder defined by pro-inflammatory markers such as rheumatoid arthritis, osteoarthritis, topical dermatitis, psoriasis, allergy and obesity.

The invention provides the use of the physiologically acceptable composition according the invention for the preparation of a product to maintain or improve gut health or gastrointestinal functioning.

The invention will now be illustrated by the following examples, which are provided by way of illustration and it will be understood that many variations in the methods described and the amounts indicated can be made without departing from the spirit of the invention and the scope of the appended claims.

### EXAMPLES

### Example 1: Anti-Inflammatory Effect on Gut Epithelium Cells

### Materials and Methods

All yeasts were produced from a non-GMO yeast strain. The yeasts included in the experiment were S. boulardii DSM 33954 (as deposited at DSMZ, German Collection of Microorganisms and Cell Cultures, Germany), available from ABBiotek as ABB1; S. cerevisiae Y1529 (as deposited at ATTC), available from ABBiotek as ABB6 in zinc enriched form; and K. marxianus V21/012435 (as deposited at the National Measurement. Institute, Port. Melbourne Vic 3207, Australia), available from ABBiotek as ABB7. The combination of these yeasts, as used herein, is also available from ABBiotek as ABB22.

The fermentation process produced a primary grown, yeast whose growth occurs under aseptic, aerobic conditions. During fermentation, the temperature, pH, and growth rate were closely regulated. In the case of S. cerevisiae, zinc sulfate was added to the yeast cream at the end of the fermentation process to a concentration of about 10% based on dry weight. The resulting product, or yeast cream was held in refrigerated storage to maintain cell viability. Prior to spray drying, the chilled yeast cream was treated with a high temperature sterilization system to obtain a tyndallized version of the yeasts. Maltodextrin or other bulking agents can be used as support agent to have standardized concentrations.

The three heat-inactivated yeasts were premixed, followed by a homogenization step. For the in vitro studies described below, the mixture was made from stock solutions of each yeast.

The yeast concentration of each yeast stock was measured by flow cytometry. A sample standardized at 1×10⁷ cells/mL in cell culture medium was prepared for each yeast. To obtain the combinations, samples of each yeast strain were mixed at a homogeneous ratio of each strain, i.e. each of the three strains in the mix were at a concentration of 0.333×10⁷ cells/ml.

In vitro immune modulating activity of yeasts and combinations thereof was studied by chemokine production by Caco-2 cells in the presence and absence of a pro-inflammatory stimulus. Caco-2 cells were cultured to confluence in 96 well plates. At the start of the experiment, cells were washed once with antibiotic free culture medium. Monolayers were incubated with test components in triplicate for 1 hour at 37° C. in antibiotic free medium. After that, the cells were incubated with medium containing the test components and 50 μg/ml gentamicin (Invitrogen), in duplicate. One of the duplicates was further stimulated with a mixture of recombinant TNFα (10 ng/ml) and recombinant IFNγ (5 ng/ml) (R&D systems), as pro-inflammatory stimulus (FIGS. 4-6). As blank control, medium only was used, without stimulation in case of FIGS. 1-3, a medium under a pro-inflammatory stimulus (a mixture of recombinant TNFα (10 ng/ml) and recombinant IFNγ (5 ng/ml) for FIGS. 4-6.

Supernatants were collected 24 h after stimulation and stored at 20° C. A Bio Plex assay (BioRad) was used to measure the IL-8, IP-10 and MCP-1 levels, according to the manufacturers' protocol.

Metabolic activity of the cells for confirming non-cytotoxicity of the test components, was analysed by WST-1 assay (Roche), according to the manufacturers protocol, after collecting the culture supernatants. The cells were found not to be metabolically active, indicating that any observed effect is not due to metabolic alterations or by cytotoxicity.

As in the other examples, one-way ANOVA was performed after which statistical differences between the control condition and the test conditions were calculated by using Dunnett's post hoc test. Significance thresholds as used in the figures were as follows: * p<0.05, ** p<0.01 and *** p<0.001.

### Results

FIG. 1 depicts the in vitro IP-10 chemokine production by Caco-2 cells after incubation with 3 yeasts and a combination thereof, in the absence of a pro-inflammatory stimulus

A synergistic reduction of pro-inflammatory cytokines in gut epithelial cells was in particular observed for the yeast composition comprising S. boulardii, S. cerevisiae and K. marxianus, from now on called composition ABB C22, compared to the individual yeasts (FIGS. 2-3). A synergistic reduction of pro-inflammatory cytokines in gut epithelial cells after a pro-inflammatory challenge with TNFα/IFNγ was observed for the yeast composition ABB C22 compared to the individual yeasts (FIG. 4-5). Furthermore, it is observed that the composition ABB C22 outperforms a composition only comprising the yeasts S. boulardii and S. cerevisiae (FIGS. 2-6), indicating a crucial role of K. marxianus in obtaining a positive effect for several marked, which is even synergistic in at least some aspects.

### Example 2: Anti-Inflammatory Effect on Immune Cells

### Materials and Methods

The yeasts and combinations thereof were prepared as in example 1.

In vitro immuno-modulating activity of yeasts and combinations thereof was studied by following the cytokine production by THP-1 cell line (macrophages). The human THP-1 cell line was cultured 1×10⁵ cells/well in 96-well plates in the presence of 100 nM phorboll2-myristate 13-acetate (PMA, Sigma) and incubated for 48 hours to induce differentiation of the THP-1 monocytes into macrophages. Cells were washed and incubated for another 72 hours in culture medium. After this, the cells were incubated for 1 hour with the test components, after which the cells were incubated for another 16 hours with and without LPS (100 ng/ml, Sigma) in the presence of the test components. All conditions were tested in triplicates.

Supernatants were collected after stimulation and stored at −20° C. An ELISA assay (IL-10 Human Uncoated ELISA Kit, TNF-α Human Uncoated ELISA Kit, Life Technologies) was used, to measure the TNF-α and IL-10 levels, according the manufacturers protocol. TNF-α/IL-10 ratio was calculated as a measure of anti-inflammatory effect of the tested components.

Metabolic activity of the cells, for confirming non-cytotoxicity of the test components, was analysed by WST-1 assay (Roche), according to the manufacturers protocol, after collecting the culture supernatants. The cells were found not to be metabolically active.

### Results

Reduction of TNFα/IL-10 ratio, as a measure of anti-inflammatory effect, in immune cells was observed for the yeast composition ABB C22 compared to the individual yeasts and compared to a composition only comprising the yeasts S. boulardii and S. cerevisiae (FIG. 7).

### Example 3: Intestinal Barrier Integrity after Challenge

### Materials and Methods

The yeasts and combinations thereof were prepared as in example 1.

The effect of the yeasts and combinations thereof on gut barrier function upon a challenge was studied by following trans-epithelial electric resistance (TEER) over a gut cell layer.

Caco-2 cells were seeded (2×10⁴ cells/cm2) and cultured on Transwell polycarbonate cell culture inserts with a mean pore size of 0.4 μm and a diameter of 0.33 cm² (Greiner Bio one) until full differentiation 1000Ω). As indicative measure of barrier integrity, TEER was measured with an EVOM2 epithelial volt ohmmeter, (World Precision Instruments).

At the day of the experiment, the cells were washed and incubated for 1 hour at 37° C. with antibiotic- and serum-free medium containing the test components. Subsequently, the wells were exposed for 6 hours to ETEC H10407 (MOI 200:1) in the presence of the test components. TEER was measured before the start of the experiment (t=1), 1 hour after exposure to the test components and before addition of the ETEC (t=0), and 1 h, 2 h, 3 h, 4 h and 6 h after exposure to ETEC (respectively t=1, t=2, t=4 and 6 h).

The TEER values of the individual conditions after exposure to the pathogens were related to their own TEER value at t=0 and expressed as Δ TEER (Ω·cm2). A negative control (ETEC H10407 only) and a positive control which was not exposed to the pathogen nor to test components was included. All conditions were tested in triplicate.

Transepithelial flux with FITC Dextran (Sigma) was measured at various timepoints after the TEER measurement.

### Results

Protection of the gut epithelium integrity was measured after incubation with an infective agent known to disrupt the epithelium monolayer, here E. coli ETEC. The yeast combination ABB C22 has a higher increase in TEER relative to the negative control, compared to the individual yeasts or the combination of S. cerevisiae with S. boulardii after 1 and 2 hours of incubation (FIG. 8).

### Example 4: Intestinal Barrier Integrity Formation

### Materials and Methods

The yeasts and combinations thereof were prepared as in example 1.

The effect of yeasts and combinations thereof on growth and differentiation of intestinal epithelial cells was followed during formation of the gut cell monolayer.

Caco-2 cells were seeded (2×10⁴ cells/cm2) on Transwell polycarbonate cell culture inserts with a mean pore size of 0.4 μm and a diameter of 0.33 cm² (Greiner Bio one). After overnight attachment of the cells, the test components were added at the apical side of the cells.

The test ingredients were prepared and stored at 20° C. in aliquots. Every two days a new aliquot was taken to refresh the ingredients. To avoid overgrowth of epithelial cells by the tested yeasts (when able to grow in aerobic conditions), 10% of conditioned medium of the yeasts and combinations thereof, together with the heat-killed yeasts were used.

As indicative measure of cell growth and barrier integrity formation, TEER was measured every other 2 days, with an EVOM2 epithelial volt ohmmeter (World Precision Instruments).

### Results

Increase in TEER was used to measure the spontaneous formation of the gut epithelium over time. An increase in TEER is observed when compared to the negative control for the three yeasts after 16 days, and maintained until day 20 only for S. cerevisiae (FIG. 9a, top). In contrast, the increase in TEER with respect to the control from day 16 observed for the combination ABB C22 is maintained and increased until day 22 (FIG. 9b, bottom).

A higher slope for TEER increase, indicative for a faster TEER increase, is observed for the combination ABB C22 compared to individual yeast strains and the combination of S. cerevisiae and S. boulardii (slopes of trendlines in FIG. 10).

1. A physiologically acceptable composition comprising (i) at least one component selected from the group consisting of S. boulardii yeasts, S. boulardii lysates, S. boulardii cell wall components, and S. boulardii extracts, further comprising (ii) at least one component selected from the group of S. cerevisiae yeasts, S. cerevisiae lysates, S. cerevisiae cell wall components and S. cerevisiae extracts and further comprising (iii) at least one component selected from the group consisting of K. marxianus yeasts, K. marxianus lysates, K. marxianus cell wall components and K. marxianus extracts, wherein the S. boulardii-based fraction of the composition is 0.05-99.95 wt. % based on total yeast components, wherein the S. cerevisiae-based fraction of the composition is 0.05-90 wt. % based on total yeast components, and wherein the K. marxianus-based fraction of the composition is 0.05-99.95 wt. % based on total yeast components.

2. The physiologically acceptable composition according to claim 1, comprising S. boulardii yeast, S. cerevisiae yeast and K. marxianus yeast, wherein at least part of said yeasts is inactivated, preferably heat-killed, more preferably tyndallized.

3. The physiologically acceptable composition according to any of claims 1-2, comprising S. boulardii yeast, S. cerevisiae yeast and K. marxianus yeast, wherein at least part of one or more of said yeasts is alive.

4. The physiologically acceptable composition according to any of the preceding claims, wherein the S. boulardii is present in a concentration of at least 10⁶ cells per gram (based on total weight of the yeast components), preferably 10⁹ cells per gram (based on total weight of the yeast components) to 4×10¹⁰ cells per gram (based on total weight of the yeast components), wherein S. cerevisiae is present in a concentration of at least 10⁶ cells per gram (based on total weight of the yeast components), preferably from 10⁶ cells per gram (based on total weight of the yeast components) to 5×10⁹ cells per gram (based on total weight of the yeast components) and wherein K. marxianus is present in a concentration of at least 10⁶ cells per gram, preferably 10⁶ cells per gram (based on total weight of the yeast components) to 5×10⁹ cells per gram (based on total weight of the yeast components).

5. The physiologically acceptable composition according to any of claims 1-4, wherein at least one of said yeasts is a mineral enriched yeast, preferably a zinc enriched yeast, in particular a zinc enriched S. cerevisiae.

6. The physiologically acceptable composition according to any of claims 1-5, wherein S. boulardii is selected from the group consisting of S. boulardii CNCM I-745, S. boulardii Hansen CBS 5926, S. boulardii BLD-3, S. boulardii CCTCC M2012116, S. boulardii CNCM I-1079, S. boulardii ATCC MYA-796, S. boulardii Unique28, S. boulardii Kirknanr, S. boulardii Unisankyo, S. boulardii DSM 33954 and S. boulardii CNCM I-3799, wherein S. cerevisiae is selected from the group consisting of S. cerevisiae Y1529, S. cerevisiae CNCM I-3856, S. cerevisiae S288C and S. cerevisiae UFMG 905, and wherein K. marxianus is selected from the group consisting of K. marxianus AS41, K. marxianus B0399, K. marxianus CIDCA 8154, K. marxianus CBS1553, K. marxianus M3, K. marxianus V21/012435 and K. marxianus Z17.

7. The physiologically acceptable composition according to claim 6, wherein S. boulardii is S. boulardii S. boulardii DSM 33954, wherein S. cerevisiae is S. cerevisiae S288C or S. cerevisiae Y1529; and wherein K. marxianus is K. marxianus V21/012435.

8. The physiologically acceptable composition according to any of claims 1-7, wherein the S. boulardii-based fraction of the composition is 5-95 wt. % based on total yeast components, wherein the S. cerevisiae-based fraction of the composition is 10-90 wt. %, preferably 10-80 wt. %, based on total yeast components, and wherein the K. marxianus-based fraction of the composition is 5-95 wt. % based on total yeast components, with the proviso that the total of said three fractions is 100 wt. % or less.

9. The physiologically acceptable composition according to any of claims 1-8, wherein the S. boulardii-based fraction of the composition is 15-50 wt. % based on total microbiological material of the composition, wherein the S. cerevisiae-based fraction of the composition is 15-50 wt. % based on total microbiological material of the composition, and wherein the K. marxianus-based fraction of the composition is 15-50 wt. % based on total microbiological material of the composition, with the proviso that the total of said three fractions is 100 wt. % or less.

10. The physiologically acceptable composition according to any of claims 1-9, wherein the composition is a pharmaceutical product, a nutraceutical product or a cosmeceutical product.

11. The physiologically acceptable composition according to any of claims 1-9, wherein the composition is a nutritional product, preferably a product selected from the group consisting of dairy products, infant formulae, fruit-based products, cereal products, snacks, vegetable beverages, smoothies and isotonic drinks.

12. The physiologically acceptable composition according to any one of the preceding claims, wherein the composition is an oral rehydration salt.

13. The physiologically acceptable composition according to any of the preceding claims for use in the treatment of a human or animal by therapy.

14. The physiologically acceptable composition according to any of claims 1-13, for use in maintaining or improving of gut health or gastrointestinal functioning in an individual.

15. The physiologically acceptable composition according to any of claims 1-14, for use in the treatment of a gastrointestinal disorder.

16. The physiologically acceptable composition for use according to claim 15, wherein the gastrointestinal disorder involves impairment of the gut barrier function.

17. The physiologically acceptable composition for use according to claim 15 or 16, wherein the gastrointestinal disorder is selected from the group consisting of inflammatory bowel disease (IBD) and irritable bowel syndrome (IBS).

18. The physiologically acceptable composition for use according to claim 15, 16 or 17 in the treatment of diarrhea.

19. The physiologically acceptable composition for use according to claim 18, wherein the diarrhea is bacterially induced diarrhea.

20. The physiologically acceptable composition according to any of claims 1-19, for use in the treatment of a disorder defined by pro-inflammatory markers, such as rheumatoid arthritis, osteoarthritis, topical dermatitis, psoriasis, allergy or obesity.

21. The physiologically acceptable composition for use according to any of the claims 13-20 wherein the composition is to be administered administration into the gastrointestinal tract, preferably orally, or wherein the composition is to be administered topically.

22. The physiologically acceptable composition for use according to any of the claims 13-21, wherein the composition is for use in the treatment of a human.

23. Use of a physiologically acceptable composition according to any of the claims 1-12 as a food additive, a feed additive, a functional food in human nutrition, a functional food in animal nutrition, a food additive or functional ingredient for nutraceuticals.

24. Use of a physiologically acceptable composition according to any of the claims 1-12 as a probiotic, a postbiotic, a paraprobiotic, a prebiotic, a symbiotic or a probiotic-substitute.

25. A medical device comprising the physiologically acceptable composition according to any of the claims 1-12.

26. Medical device according to claim 25, wherein the device is selected from the group of patches and plasters.

27. S. boulardii yeast (as deposited at DSMZ, German Collection of Microorganisms and Cell Cultures, Germany)having deposit number DSM 33954.
